(19) United States
(12) Patent Application Publication
(10) Pub. No.: US 2007/0265633 A1
(43) Pub. Date: Nov. 15, 2007

(54) Implement and method to extract nucleus from spine intervertebral disc

(76) Inventors: Jon Kenneth Moon, Edina, MN (US); Thomas John McPeak, Shakopee, MN (US); Robert Conner, Plymouth, MN (US); Partrick Corneille, Minneapolis, MN (US)

Correspondence Address: BECK AND TYSVER P.L.L.C., 2900 THOMAS AVENUE SOUTH, SUITE 100, MINNEAPOLIS, MN, 55416, US

(21) Appl. No.: 11/800,828

(22) Filed: May 8, 2007

Related U.S. Application Data
(60) Provisional application No. 60/747,089, filed on May 11, 2006.

(51) Int. Cl. A61B 17/00

(52) U.S. Cl. 606/83

(57) ABSTRACT

This invention proposes a device directed to rapid surgical removal of the nucleus pulposus from the spine intervertebral space. The invention is manipulated within the intervertebral space to engage and dislodge small pieces of nucleus material that are mobilized proximally for disposal. Aspects of the invention are included to protect the endplate tissue of vertebrae and limit damage to the integrity of the disc annulus.

### CROSS REFERENCE TO RELATED APPLICATION

This utility application claims the benefit of U.S. Provisional Patent Application Ser. No. 60/747,089, filed May 11, 2006, incorporated by reference herein in its entirety.

### FIELD OF THE INVENTION

This invention relates to devices and methods for use in interventions to restore spinal function. More specifically, the invention removes nucleus pulposus from the intact spine intervertebral disc during surgical therapy to treat herniation or degenerated discs.

### BACKGROUND OF THE INVENTION

Back and spinal ailments trouble thousands of Americans every year. In 2003 approximately 11 million people had impaired movement because of back pain, resulting in $80 billion of lost work and productivity. Back pain is a top cause of health care expenditures, amounting to $50 billion in the USA alone. However, only 2 percent of patients seek current implant therapies that create spinal fusion, and they typically do so only at an advanced stage of disease.

Disc degeneration is part of the natural process of aging and has been documented in approximately 30% of 30 year olds. As the population ages, it is even more common for individuals to have signs of disc degeneration. Disc degeneration is an expected finding over the age of 60.

Many back problems result from failure of the annulus (also called the disc annulus or outer fibrous ring) and from herniation of the nucleus pulposus (also called the disc nucleus) through the annulus of the intervertebral disc to compress the spinal cord or nerve roots. Currently, there are only limited treatments for these ailments. First, if the nucleus is still relatively intact, a physician can remove the herniating portion and leave the remaining nucleus in an effort to maintain the integrity and mobility of that spinal region. Successful surgery depends on integrity of the annulus and involves the assessed risk of additional future herniation. Or, physicians can remove much of the intervertebral disc with the intention of preventing future herniations by facilitating a fusion of adjacent discs.

These interventions are great advancements over treatments that were available just decades ago. But, they introduce several concerns and difficulties. One of the most difficult decisions that physicians face is to determine the amount of nucleus to remove. If too much is removed then mobility can be reduced, too little and the herniation may recur. There is also substantial risk of damage to the annulus that could impair healing. Procedures that remove the complete intervertebral disc, discectomy, damage the vertebral end plate. Due to the similar texture of the ligamentum flavum and the dura there is also concern of cutting into the dura, which could result in neurological complications. Finally, these procedures produce large amounts of scarring, which limits the scope of revision surgeries.

A new treatment uses intervertebral implants to replace the nucleus with materials that restore mobility and avoid adjacent segment deterioration without the risk of herniation. Manufacturers have developed implants to the point that several forms of the prostheses are in clinical trials. Although there are associated problems and difficulties, these implants are poised to be a major breakthrough treatment of failed intervertebral discs, particularly in young people. The implants are placed within the space defined by the annulus after as much of the nucleus as possible has been removed. Because the goal of the surgery is to restore mobility, the annulus, vertebral endplates and other disc structures must be undamaged.

Presently, most disc surgeries involve partial removal of the nucleus pulposus (nuclectomy). Or the nucleus is removed along with the entire intervertebral disc (discectomy). Standard surgical tools, such as curettes, bone nibblers or pituitary rongeurs, and a variety of techniques have been adapted for these procedures. All of these prior art tools were designed for purposes other than spinal surgery and are poorly suited to nucleus removal, especially when other tissues must be spared from injury. Generally, surgeons have experience and training only for procedures that require incremental extraction of small pieces of the nucleus (micro or partial nuclectomy). When applied to complete nuclectomy these tools lack the flexibility and control to remove all of the nucleus and invariably cause damage to the surrounding annulus fibrosus and vertebral end plates. In addition, substantial skill and dexterity is required to produce satisfactory results. Even in the hands of an experienced surgeon, nucleus extraction can be the most prolonged and difficult stage of the newer forms of spinal surgery.

No devices or methods have been developed specifically to remove the entire nucleus while minimizing trauma to other tissues. Maintaining the integrity of surrounding tissue is necessary to hold the implant in place and allow proper support and separation of the surrounding vertebrae. Some the implants will function poorly or risk new herniation if 20% or even as little as 10% of the original nucleus is left behind. A clean bed, free of nuclear material in critical locations, within which to deploy or graft the implants will also be crucial to the success of surgery. As a result, special methods, tools, or procedures are needed that can cleanly remove the nucleus without damaging the fibers of the annulus.

In an effort to address some of these limitations, physicians and researchers are searching for new methods of treatment for the herniated nucleus pulposus. They are looking at treatments that restore the function of the nucleus, regenerate the structure of the annulus, or are implanting artificial discs. Each of these proposed treatments introduces new difficulties and will need additional support mechanisms to prepare for the procedures. One of the most promising therapies is nucleus replacement. It is superior to traditional disc fusion because it restores movement and function to the disc space. It also promises to be superior to artificial disc implantation because much more of the original tissue is preserved, the procedure is faster, and there is less risk of malpositioning. Neither fusion nor artificial disc implantation are likely to ever be compatible with percutaneous access and thus carry a greater risk of infection and damage to other tissues or organs.

Most approaches to nucleus replacement will require removing the entire nucleus. There are few methods of removing the nucleus to prepare for nucleus replacement. These include the use of manual surgical implements such as curettes, bone nibblers, and pituitary rongeurs. The procedure involves incremental extraction of small pieces of the damaged portion until a the surgeon judges that a sufficient amount has been removed.

There are few companies currently looking at methods for removal of the nucleus pulposus, as nucleus replacement is a fairly new treatment modality. Clarus Medical has developed the ‘cut and suction’ method of percutaneous discectomy. Their product is the Nucleotome, a mechanical device with a blunt drill passing through a cannula that enters the disc site. It uses a rounded tip, shaped like a blunt drill to decrease the risk of cutting into the annulus. Stryker Corporation offers another rigid design, the “Dekompressor”, a percutaneous discectomy probe. It has a battery-operated disposable hand piece attached to a helical probe. The cannula allows access to the disc space, and the probe rotates and removes nucleus material through a suction mechanism. Both devices are too stiff to easily remove all of the nucleus

ArthroCare Corporation, has worked on coblation technology, which involves the use of low energy radio-frequency waves. This energy creates an ionic plasma field from the sodium atoms found in the nucleus. A molecular dissociation process occurs due to this low temperature plasma field, which converts this tissue into gases that exit the treatment site. The product is named the Spine Wand. It acts as drill as it is advanced into the disc. The tissue is converted into gas that exits the disc through the cannula. An accessory to the Spinal Wand is the System 2000 Controller. This accessory uses a combination of ablation, resection, coagulation and suction. A bipolar cautery is employed. However, the insertion depth up to the annulus must be predetermined and the wand is difficult to steer to remote parts of the nucleus space.

Laser discectomy employs laser energy to vaporize portions of a diseased disc. It is compatible with through minimally invasive surgery. However, laser techniques are generally useful to remove only small amounts of material because of the heat generated and other limitations. In addition, vaporized material expands to a gaseous phase and must be removed.

This invention proposes devices and methods directed to improving complete removal of the disc nucleus. The new process must be a relatively quick and cost effective alternative to current procedures. In addition, the new method or device must facilitate a complete and clean removal of the disc in a safe manner that does not compromise the integrity of the annulus.

### OBJECTS OF THE INVENTION

An object of the present invention is to overcome the drawbacks described above and other limitations in existing systems by providing a surgical device to remove almost the entire nucleus from a spinal intervertebral disc.

Another object of the invention is to remove nucleus material with minimal or no damage to surrounding tissues or structures such as the disc annulus, vertebral endplates, spinal nerves or blood vessels.

Another object of the invention is to be minimally invasive and carry a low risk of infection or discomfort to the patient.

Another object of the invention is to provide a system and method that removes the nucleus rapidly.

Another object of the invention is to provide a system and method that allows a surgeon to remove the nucleus without prolonged training, practice or skill.

Another object of the invention is to provide a system and method that removes the nucleus while allowing the surgeon fine control of the procedure.

These and other objects of the invention are accomplished according to various embodiments of the invention.

### BRIEF DESCRIPTION OF THE DRAWINGS

FIG. 1 is a frontal-lateral view of the anatomy of a section of the human lumbar spine.

FIG. 2 is a superior view cross section of the anatomy of a human lumbar intervertebral disc.

FIG. 3 is a superior view in cross section of a herniated human intervertebral disc.

FIG. 4 is a side view representation of the human spine in the vicinity of a herniated disc.

FIG. 5 is a component view of the present invention.

FIG. 6a is close up view of the shearing type embodiment. This figure shows the present invention in the open position.

FIG. 6b is a view of the shear type embodiment in the closed position.

FIG. 6c is a side view of the shear type embodiment detailing cutting edge angles.

FIG. 7a is a close up view of twist type distal tip. This figure shows the present invention in the open position.

FIG. 7b is a view of the twist type distal tip in the closed position.

FIG. 7c is a side view of the twist type embodiment detailing cutting edge angles.

FIG. 8 is an isometric view of a shear type embodiment with a distal tip extension.

FIG. 9 is an isometric view of a punch type embodiment.

FIG. 10 is an isometric view of a reciprocating cutting loop embodiment of the present invention.

FIG. 11 is an isometric view of a reciprocating disk embodiment of the present invention.

FIG. 12 is an isometric view of a reciprocating bilobed cutting loop embodiment of the present invention.

FIGS. 13a and 13b are isometric views of a rotational cutting loop embodiment of the present invention.

FIG. 13c is a phantom isometric view of the rotational cutting loop embodiment of the present invention comprising an auger.

FIGS. 14a to 14c are isometric views of a rotational cutting vane embodiment of the present invention.

FIG. 15 is an isometric view of another rotational cutting vane embodiment of the present invention.

### DETAILED DESCRIPTION OF THE INVENTION

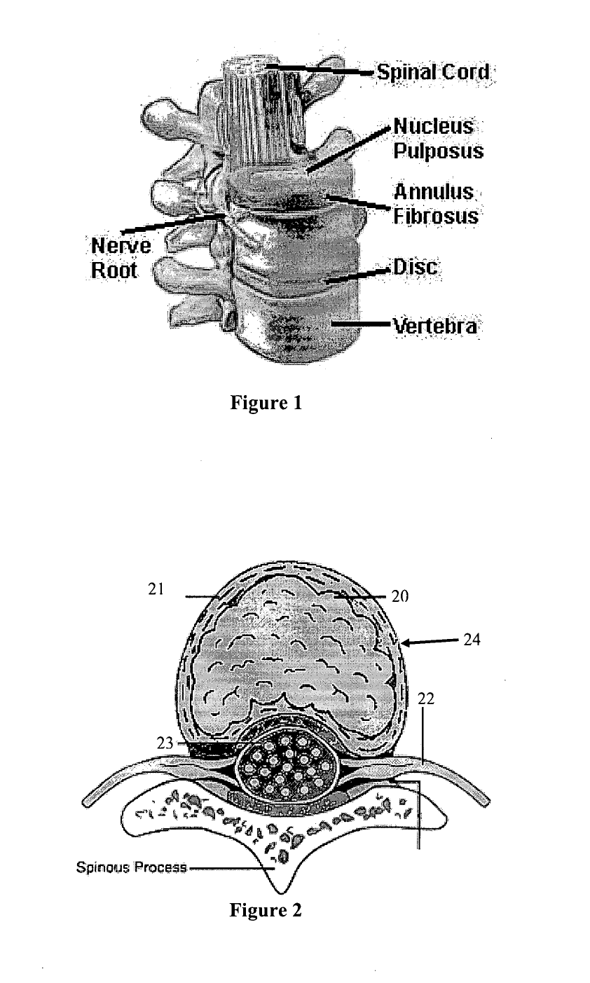

This invention overcomes various limitations of prior art means to remove nucleus pulposus from spinal intervertebral discs. FIG. 1 shows a section of the lumbar spine with major anatomic features labeled. Vertebrae are the bones that provide essential strength and stiffness to the spine and afford protection to the spinal cord, spinal nerve roots and major blood vessels (the blood vessels are not shown but are located opposite the spinal cord). The discs located between vertebra provide the spine with the ability to articulate by lubricating and separating the vertebrae.

FIG. 2 is a superior sectional view through an intervertebral disc 24 of the lumbar spine, the front of the body is upward in this view. Spinal nerves 22 radiate from the spinal cord 23, located posterior to the spine, to provide control and sensation to various segments and organs of the body. The disc 24 is roughly kidney shaped and defined by the annulus fibrosus 21. The annulus is composed of concentric layers of fibrous tissue that seal the space between vertebra located above and below the disc (not shown). Each layer of annulus 21 connective tissue is comprised of type I collagen oriented at approximately 30°. Successive annulus 21 layers alternate the 30° angle to provide substantial resistance to pressure from inside the disc 24. Within the space defined by the annulus 21 is the nucleus pulposus 20. The nucleus is avascular and comprised of hydrated mucoprotein gel and type II collagen fibers.

The intervertebral disc functions somewhat like a water bed to allow articulation of the spine. When a person is upright substantial hydrostatic pressure is developed within the disc 24 and this pressure increases at lower portions of the spine, particularly the lumbar and sacral region. The annulus 21 serves to contain nucleus 20 that is under pressures in the range of 690 to 2000 kPa (100 to 300 psi). Articulation of the spine is accommodated by displacement of nucleus material from one side of the nucleus space to another. In a normal, healthy spine the vertebrae are prevented from contacting each other even at maximal angles of articulation.

In young adults the intervertebral disc 24 is approximately 7 to 9 mm thick. With age and disease the hydration level of the nucleus 20 decreases. This thickens the nucleus from a soft gel-like consistency to become relatively stiff. Further degeneration with age and disease can occur to both the nucleus 20 and the annulus 21. This may allow the thickness of the disc 24 to decrease until, in the final stages, the vertebrae are in contact during some or all postures and movement. Contact between vertebrae damages these bony structures and generates substantial pain. Disc thickness greater than approximately 4 mm is presently considered suitable for nucleus replacement therapy. At lesser thickness treatment will usually involve removal of the disc 24 for spinal fusion or implantation of an artificial disc.

Because the nucleus 20 is avascular there are no living cells and exchange of fluids is through the cartilaginous endplates (not shown) covering the vertebral body. The endplates are a thin layer of primarily hyaline cartilage. The endplates are important to proper function of the intervertebral disc. In traditional therapies of fusion and disc replacement the endplates are not preserved so surgical techniques generally disregarded protection of the endplates. With motion restoration implantation of nucleus replacements the endplates must be protected from damage.

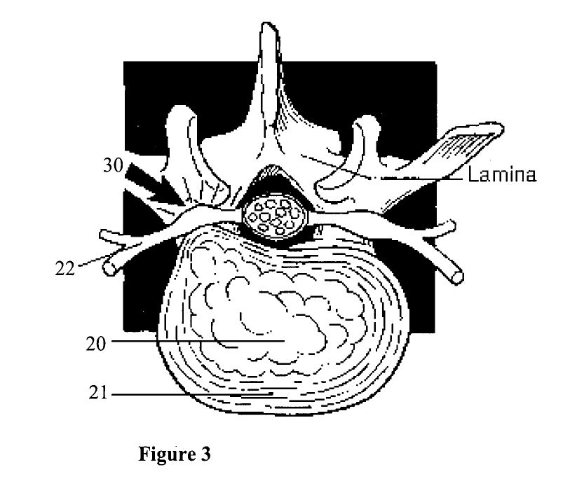

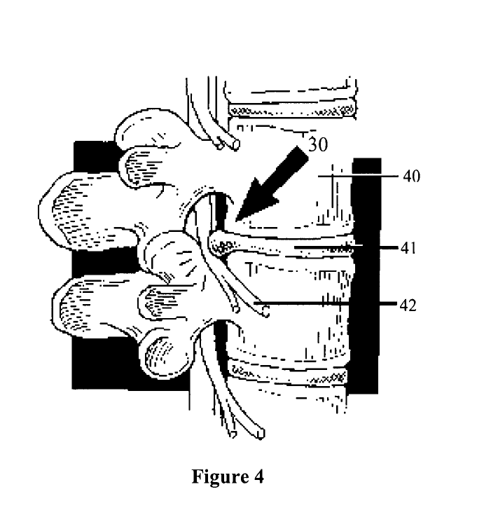

Similarly, with age and disease the annulus 21 may become weakened. This is a frequent cause of herniation, as illustrated in FIG. 3. As shown, the annulus 21 has weakened under pressure exerted by the nucleus 20 (in response to compression from the vertebrae) and compresses spinal nerve root 22. FIG. 4 is lateral view of a disc 41 herniation impacting spinal nerve 42 caused by annular failure 30. Similarly, the annulus 21 can fail such that nucleus material 20 exits the annulus and causes a direct effect on the nerve. In addition to being one of the major causes of disc therapy, degeneration of the annulus makes it vulnerable to damage during nucleus removal. The various embodiments of the present invention provide means of protecting the annulus from penetration or disruption.

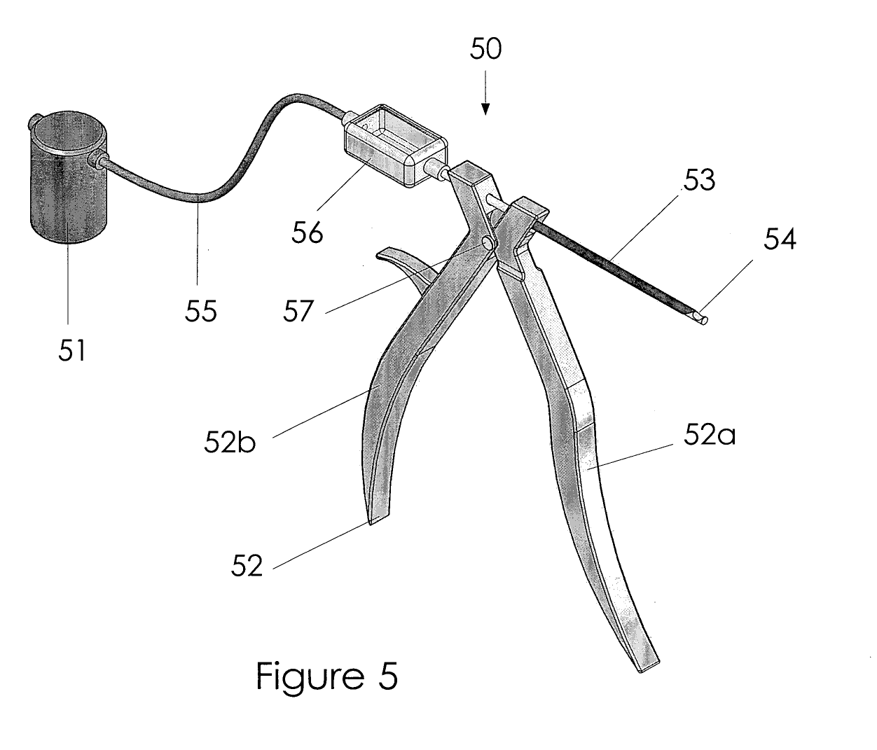

A first embodiment 50 of the present invention is illustrated in FIG. 5. It comprises a hand control 52, vacuum source 51 connected via flexible tubing 55 to a nucleus collection container 56 which in turn provides vacuum to the hollow lumen of the nucleus collection tube 54, and cutting tube 53. The cutting tube 53 has an inner diameter larger than the outer diameter of the collection tube 54 and is arranged to slide over the collection tube 54. The distal portions of the cutting 53 and collection 54 tubes (away from the vacuum source) are intended to be operated within the intervertebral disc 24 to remove nucleus material 20. The diameters of the distal portions of the tubes 53 and 54 are smaller than the height of intervertebral disc defined by the separation of the vertebrae forming the disc. These diameters are preferably less than 4 mm to allow insertion through a minimally invasive surgery guiding device and to fit within diseased or compressed intervertebral discs.

To aid in accessing and navigating the annulus space of the intervertebral disc the tubes 53 and 54 of the invention 50 may be formed of a partially elastic material that can bend through an angle up to approximately 20 degrees in the range of force that may be conveniently be applied by hand. Further, the most distal portion (e.g., a tip) of the collection tube 54 preferably comprises a material with a hardness in the same range as annulus 21, or softer. It may also be formed with a rounded or blunted surface. These will aid in protecting the annulus and vertebral surfaces from iatrogenic damage. The length of the tubes 53 and 54 is chosen to allow for use in less invasive or minimally invasive surgery. The tubes 53 and 54 are preferably manufactured of material with relatively high strength, such as stainless steel braid or polycarbonate, that resists fracture when manipulated by the operator. One or both of the tubes 53 and 54 may be formed of transparent material, depending on operator preference to observe the removal of nucleus material 20.

The collection container 56 is also preferably formed of transparent material and is sealed except for the outlet and inlet ports connected respectively to the vacuum source 51 and collection tube 53. The container 56 preferably is formed of two or more pieces or an access port that may be used to remove and preserve collected nucleus material 20; and markings or other means to allow estimation of the volume of nucleus material collected in the container. The container 56 also serves to prevent nucleus material 20 and other tissue from contaminating the vacuum source 51.

The two-piece hand control 52 is comprised of two arms 52a and 52b able to pivot at a pin joint 57. The shorter portion of the distal hand control arm 52a is attached to the cutting tube 53 while the proximal arm 52b is attached to the collection tube 54. Operating the hand control to bring the long portions of the arms together causes the cutting tube to move so that it substantially covers the distal portion of the collecting tube. The handle may further comprise a spring mechanism (not shown) that separates the arms once a force applied to bring the arms together is removed. Alternatively, the hand control may be arranged and connected to the tubes 53 and 54 so that bringing the longer portion of the arms together causes the cutting tube 53 to move proximally. Optionally, the tubes 53 and 54 may be manipulated directly or with gripping regions (not shown) without the aid of the hand control.

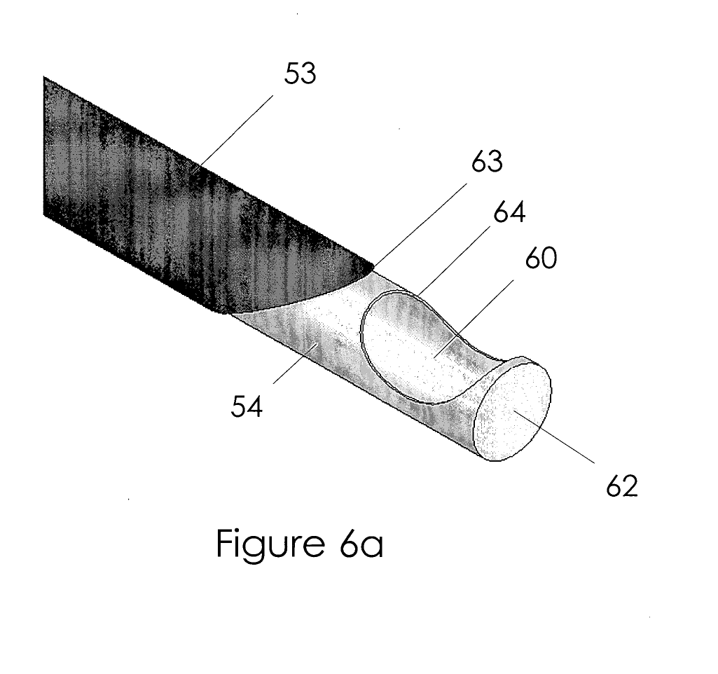

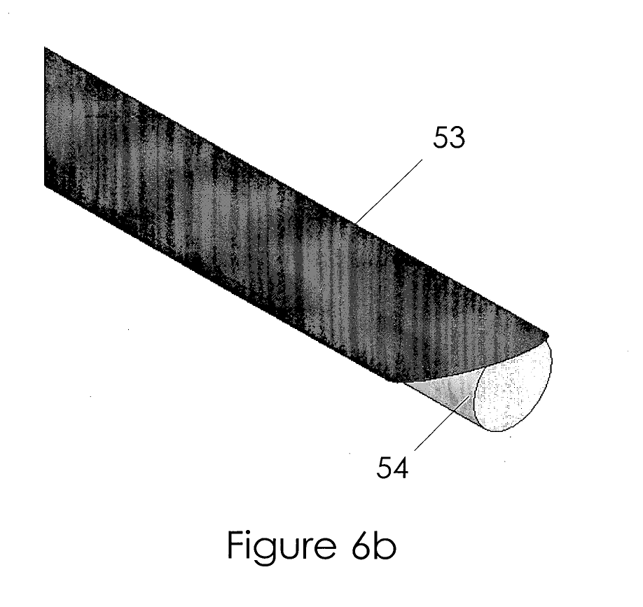

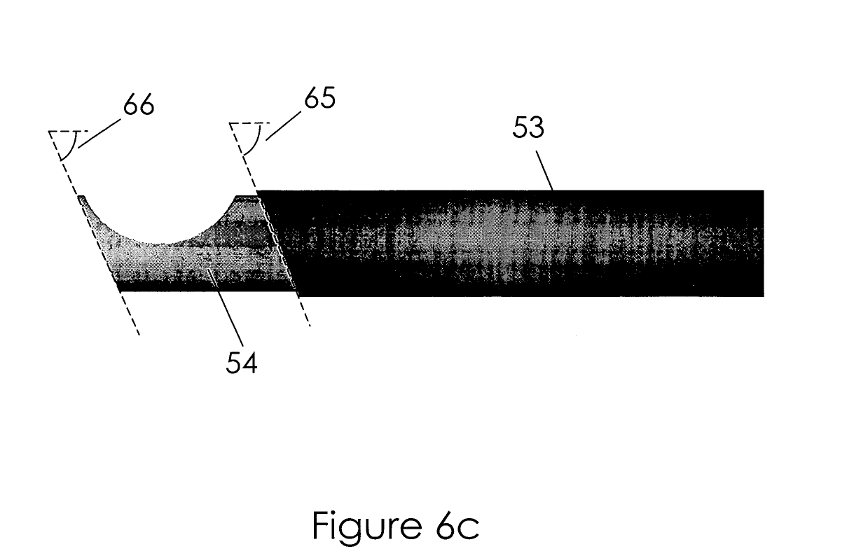

FIG. 6a is an expanded view of one embodiment of the distal portion of the invention 50. The collection tube 54 comprises a side opening 60 defined by edges 64 and end cap 62. The cutting tube 53 slides over the collection tube 54, as described above, and substantially or completely covers the side opening, as shown in FIG. 6b illustrating the closed position. The distal edge 63 of the cutting tube 53 may be thin or sharpened to be capable of penetrating and separating nucleus material 20. When the cutting tube 53 is made of a polymer or other flexible material the edge may be formed of a harder material, such as metal, attached to cutting tube by means known in the art.

The distal edge of the cutting tube 53 in the embodiment of FIG. 6a has an angle between 90 and 20 degrees, and preferably 75 to 30 degrees from the long axis of the tube. This angle preferably matches, within 10 degrees, the slope of the distal portion of edge 64 formed around opening 60 in collection tube 54. The purpose of these angles is to enhance the shearing action of the cutting tube 53 relative to the collection tube 54 in disrupting nucleus material 20. The end cap 62 of the collection tube 54 is preferably formed at an angle between 90 degrees and the angle of the cutting tube edge 63.

To remove nucleus material 20 from the intervertebral disc space 24 the distal end of collection tube 54 is inserted through an opening formed in the annulus 21. Once inside the annulus the opening 60 collection tube 54 is pushed into the nucleus material 20 so that material enters the opening 60. The cutting tube 53 is then moved forward, slicing through the nucleus material 20 and entraining a discreet quantity of nucleus material within the collection tube 54. Suction provided from the vacuum source through a lumen in the collection tube causes the entrained nucleus material to be pulled proximally and into the collection container 56. The cutting tube may be returned to a distal position immediately to re-expose opening 60 in the collection tube 54 and the collection tube repositioned to ‘pack’ more nucleus material 20 into the collection tube 54. This may aid in forming a plug of nucleus material across the entire cross-section of the lumen in the collection tube 54 so that maximum suction pressure may be developed to move the nucleus material proximally to the collection container 56. A further technique to aid in mobilizing nucleus material 20 proximally involves manipulating the cutting tube 53 across the opening 60 in the collection tube 54 to occlude air passages that may exist proximally of the nucleus material. The steps of engaging, cutting and removing nucleus material by positioning the invention 50 and moving the cutting tube 53 relative to the collection tube 54 are repeated until the desired amount of nucleus material is removed.

The operator may remove the invention from the intervertebral disc as needed to permit visualization of the annular space and then reinserted to continue the procedure. Alternatively, one or more optical fibers may be incorporated into the invention to permit visualization during nucleus removal and to aid in positioning the collection tube opening 60 for the most efficient and complete removal of nucleus material 20.

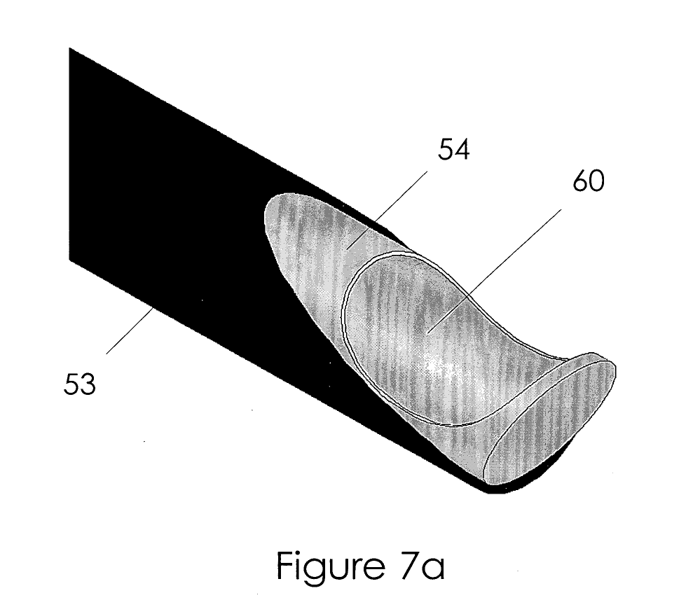

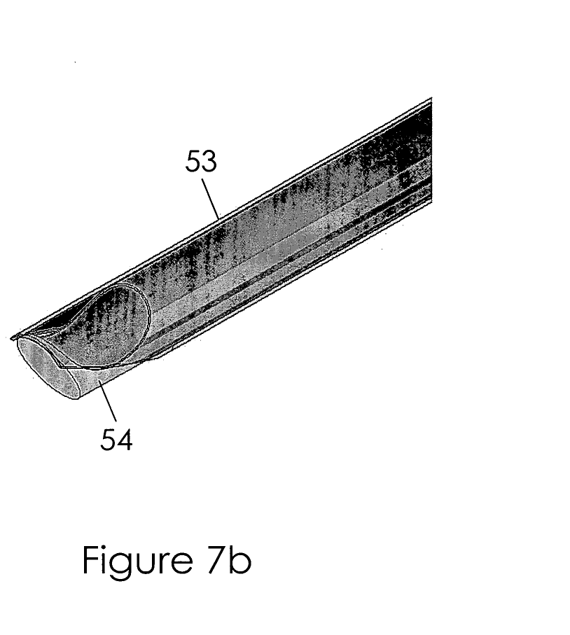

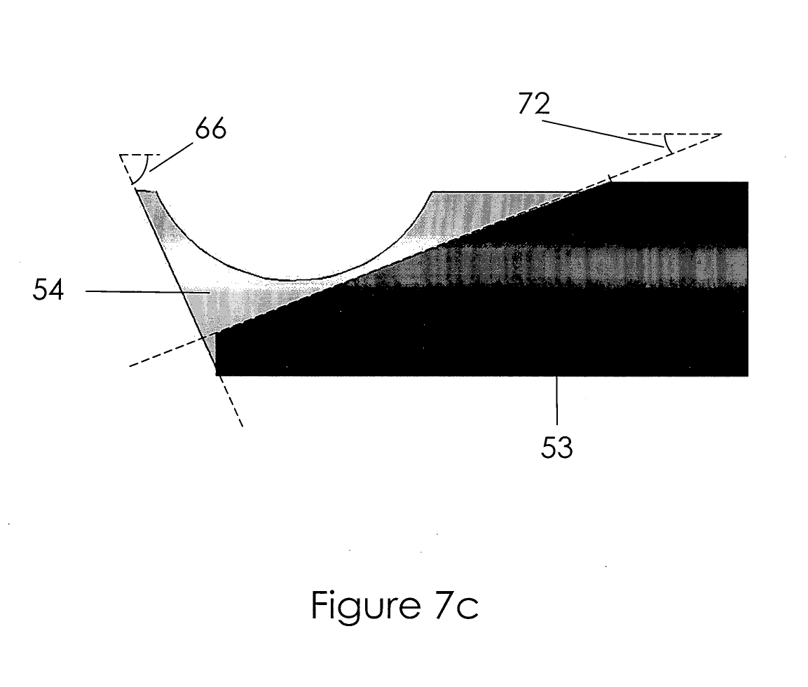

FIG. 7a shows another embodiment of the present invention wherein the cutting tube 53 is rotated around collection tube 54 to sever and entrain nucleus material 20 within opening 60.

FIG. 7b shows this embodiment with the tubes 53 and 54 in a configuration forming the closed position. The angle 72 forming the end of the cutting tube 53 is preferably 10 to 40 degrees. This smaller angle permits a larger opening 60 and a longer shearing edge. A further embodiment may combine these two modes of operation between the tubes 53 and 54: distal/proximal translation and rotation.

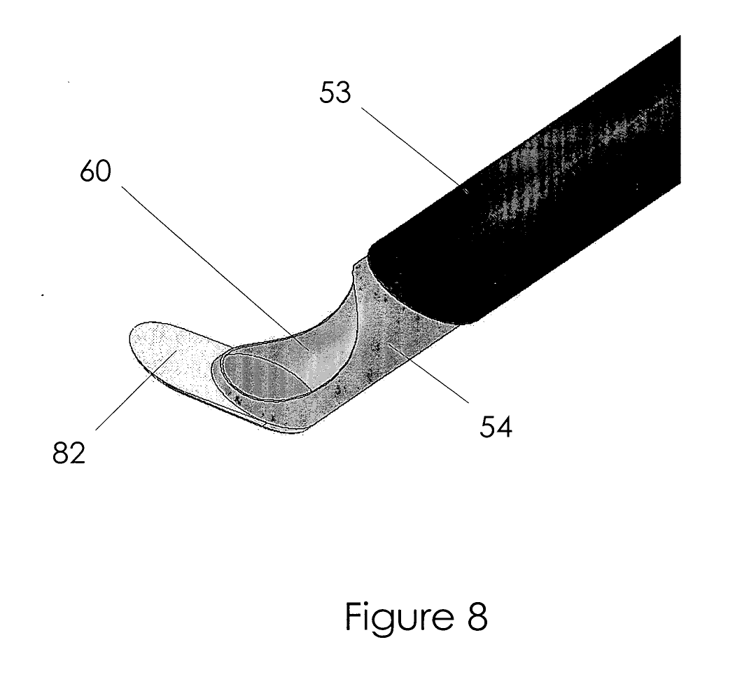

FIG. 8 illustrates a modified version of the embodiment of the invention presented in FIG. 6a. Relatively soft (compliant) material forms an extension 82 of the end cap 62 at the end of the collection tube 54. The stiffness of the extension is set sufficiently low, in the range of Shore A hardness less than 80, to protect the annulus 21 and vertebral surfaces from injury. The compliant extension comprises a long dimension, preferably at least 1.25 times the outer diameter of the collection tube 54, oriented in the same direction as the opening 60 in the collection tube 54. The width of the protruding material is approximates the outer diameter of the collection tube 54. The extension aids in disrupting or dislodging nucleus material 20 located at periphery of the annulus space of the intervertebral disc 24 and bringing the nucleus material into approximation of the opening 60.

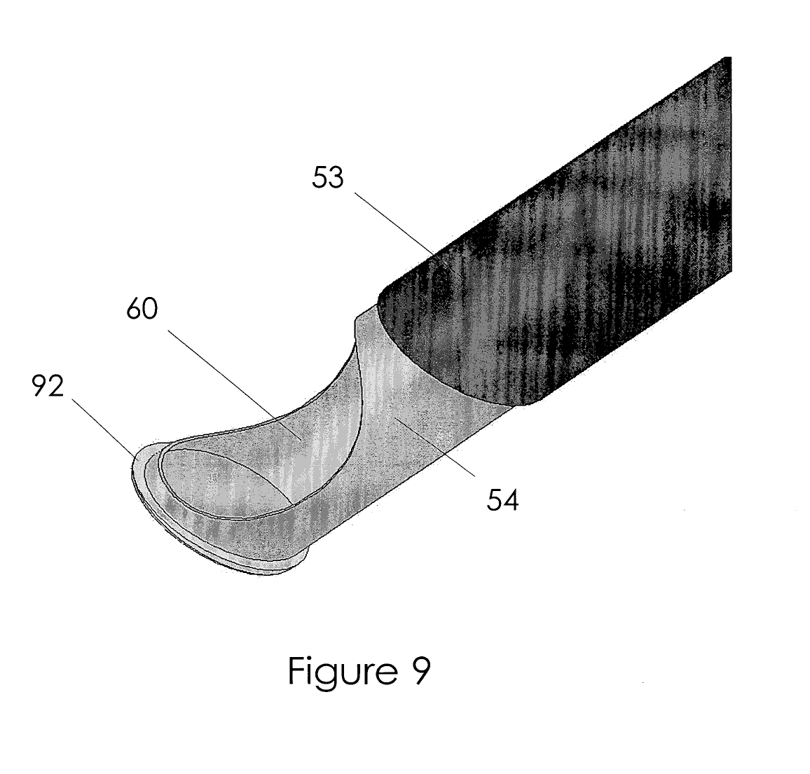

FIG. 9 shows yet another modification of the present invention. The distal cap 62 attached to the collection tube 54 incorporates an extension 92 beyond the diameter of the collection tube to approximately the outer diameter of the cutting tube 53. This embodiment permits entrained nucleus material 20 to be severed from remaining nucleus material in the fashion of a punch. The distal edge 63 of the cutting tube 53 would have the same angle 65 as the angle of the distal cap 62 and extension 92. The distal edge 63 would preferably be thin or sharpened around the entire circumference of cutting tube 53 to aid in cutting nucleus material 20. The sharpened distal edge 63 of the cutting tube 53 pressed sufficiently tightly against the extension 92 to completely sever the entrained nucleus material 20.

All of the preceding embodiments of the invention rely on force developed by suction pressure to pull entrained nucleus material 20 to the proximal end of the collection tube 54 and into the collection container 56. As described above, nucleus material becomes stiffer and is composed of increasing quantities of discreet, rigid components with age or the progress of disease. Consequently, additional features may be needed to disrupt the nucleus material and bring it out of the disc space and toward the collection container 56.

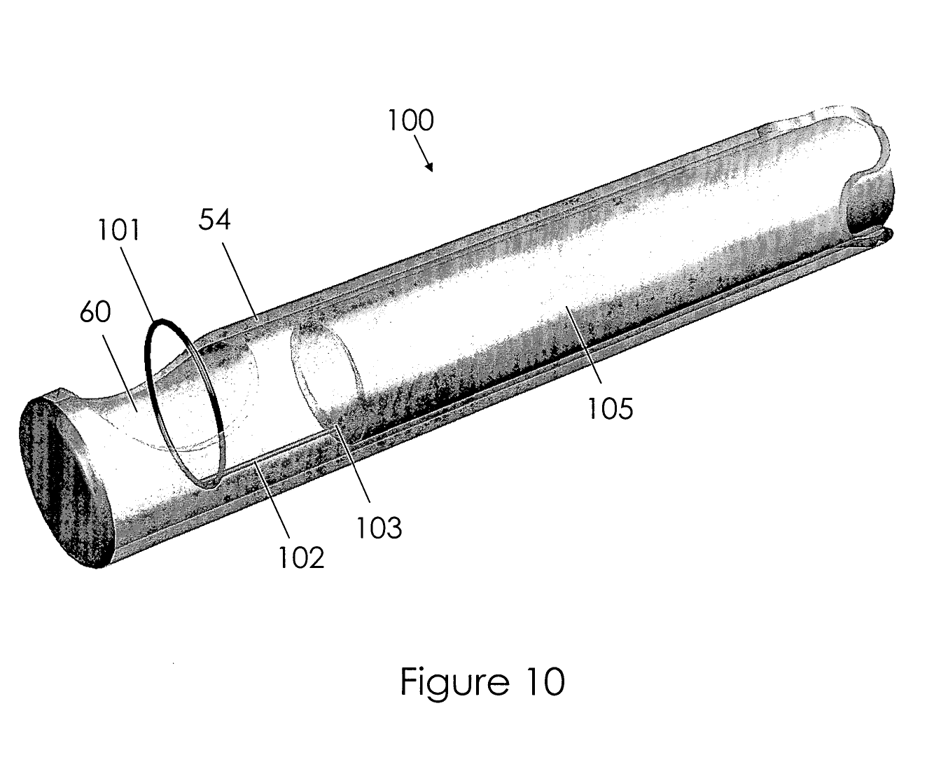

FIG. 10 illustrates an embodiment 100 of the present invention that incorporates a loop 101 attached at an angle of approximately 90 degrees to the end of a control rod 102. The loop 101 has a major diameter substantially equal to or greater than the inner diameter of the distal collection tube 54 and opening 60. The loop 101 may protrude beyond the opening 60 in the collection tube 54 and be sufficiently hard and stiff to disrupt nucleus material 20 as it is moved longitudinally within the opening 60. Preferably, the loop 101 is also sufficiently flexible to be captured entirely within the collection tube 54 without exceeding its yield stress. With the loop 101 positioned within the collection tube a cutting tube 53 (not shown) may be deployed to completely entrain nucleus material 20 within the opening 60.

The control rod 102 is manipulated by an operator from outside the intervertebral disc 24 to move the loop 101. The control rod 102 may pass through a second lumen of the collection tube 54 or a lumen 103 within a capture tube 105 located within the collection tube. Alternatively, the control rod may move freely within the main lumen of the collection tube 54. In this latter configuration the loop 101 may be withdrawn through the lumen of the collection tube 54 to assist in bringing nucleus material proximally through the collection tube. The loop 101 is used in the configuration with the capture tube 105 to bring nucleus material into the distal opening of the capture tube so that suction pressure will draw the nucleus material to the container 56 which is connected in this configuration to the capture tube instead of the collection tube 54. Alternatively, the loop 101 can trap a quantity of nucleus material 20 against the capture tube 105 and the combination withdrawn through the collection tube 54.

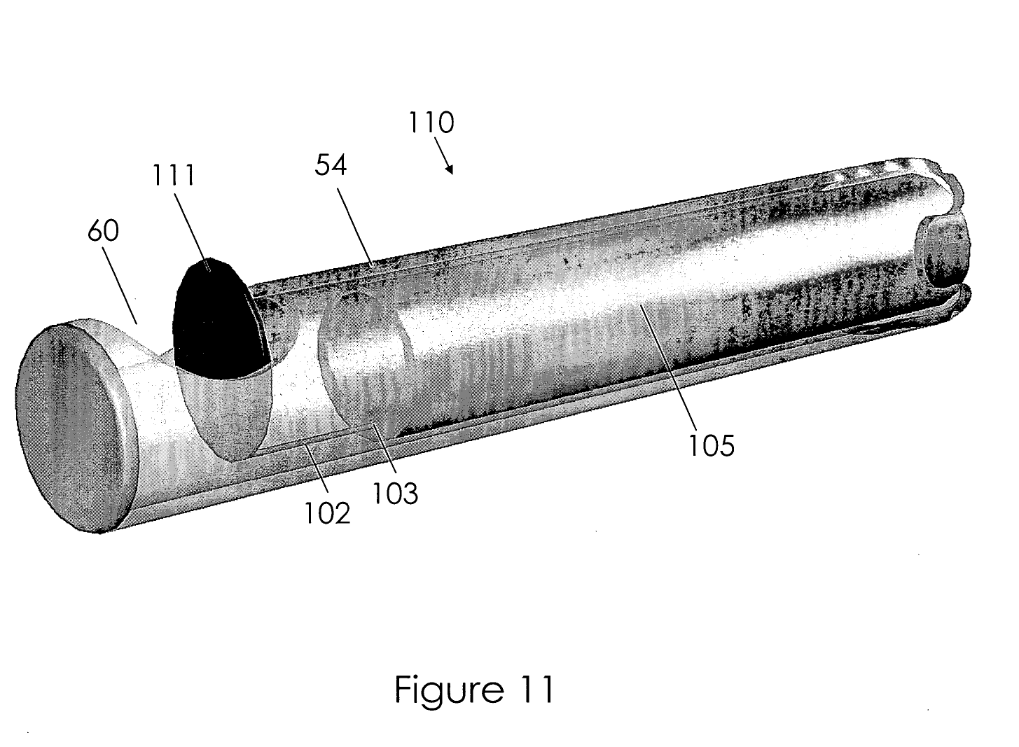

FIG. 11 shows is an alternative embodiment 110 of the invention where a solid or mesh disc 111 is attached to the end of the control rod 102. This embodiment is preferred for trapping nucleus material 20 against or within the capture tube 105.

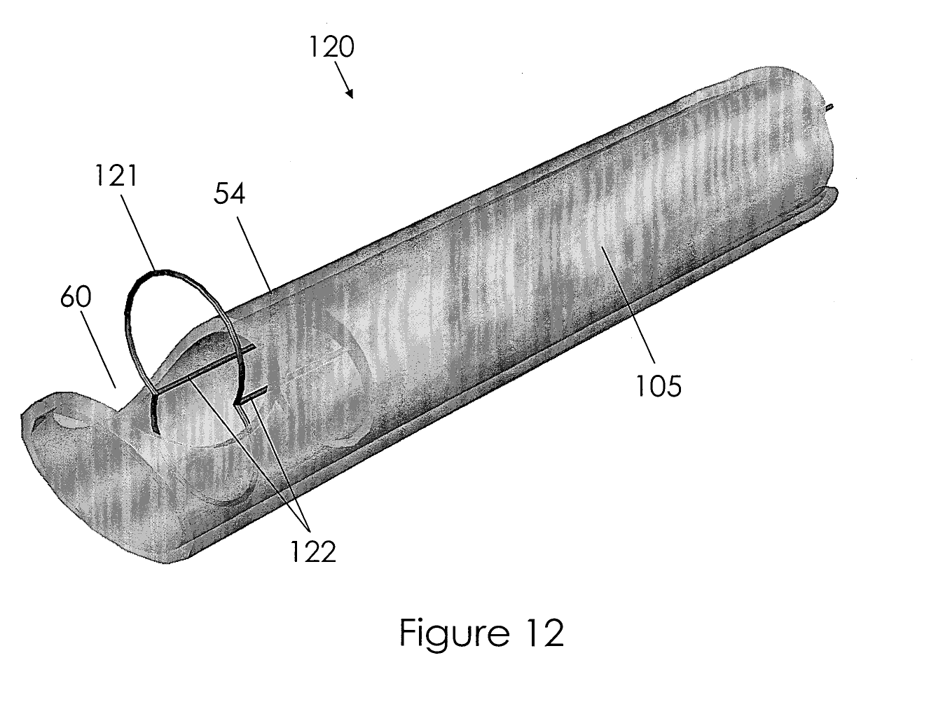

The embodiment of the present invention 120 shown in FIG. 12 comprises a plurality of control rods 122 that pass through separate lumens of the capture tube 105. Also illustrated is a loop 121 formed in a bilobed shape. One lobe of loop 121 essentially conforms to the inside diameter of the collection tube 54. The other lobe is shaped to engage more nucleus material 20 beyond the collection tube 54. Utilizing more than one control rod permits greater control of the loop 121 with less difficulty preventing unwanted rotation or bending of the loop. Functions of embodiments 110 and 100 are retained in embodiment 120.

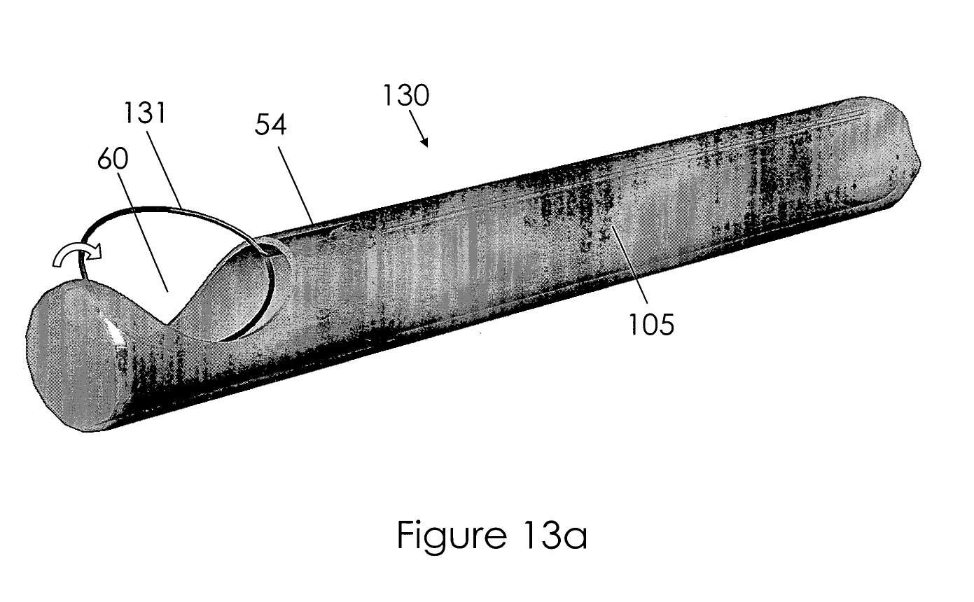

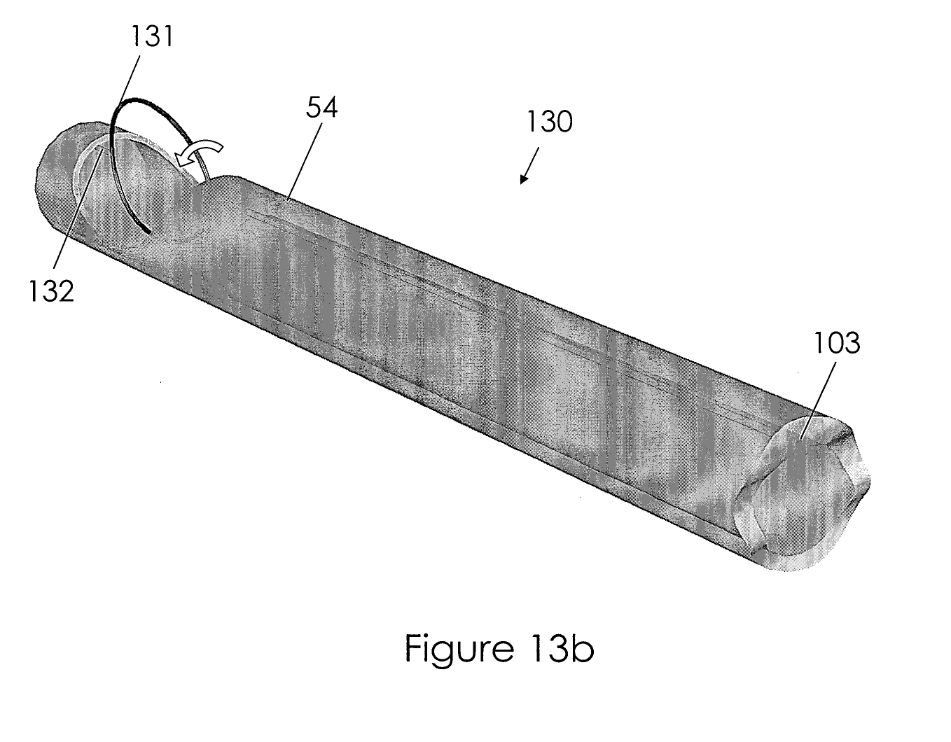

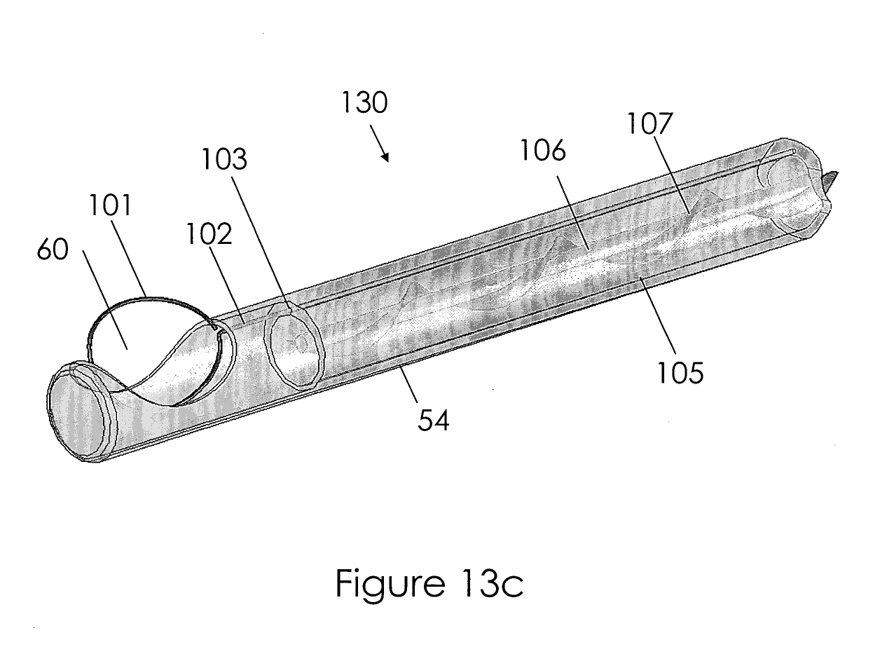

FIG. 13a shows an embodiment of the present invention comprising a loop 131 formed on the end of a rotational control rod and located in the opening 60 of the collection tube 54. The control rod passes through a lumen of the collection tube 54 near the center line of the opening 60. Alternatively, the lumen 103 guiding the control rod may be within the wall of a capture tube 105 located within the collection tube 54, as illustrated in FIG. 13b. The proximal end of the control rod is turned by the operator to cause rotation of the loop 131. This rotation disrupts portions of nucleus material that are carried into the opening 60. Suction applied to the lumen of the collection tube 54 or, if present, capture tube 105 carries nucleus material proximally in the fashion described above. FIG. 13b also illustrates a pivot extension 132 of the loop 131 that helps to stabilize the loop so that it remains within the opening 60 and does not bend when encountering stiffer nucleus material 20.

FIG. 13c shows an auger 106 located within a lumen of capture tube 105. The auger comprises and central rod and one or more flutes or vanes 107 that serve to move stiffened and granular nucleus material 20 proximally for removal. Similar auger features may be incorporated within the collection tube 54 and in any of the embodiments of the invention described herein.

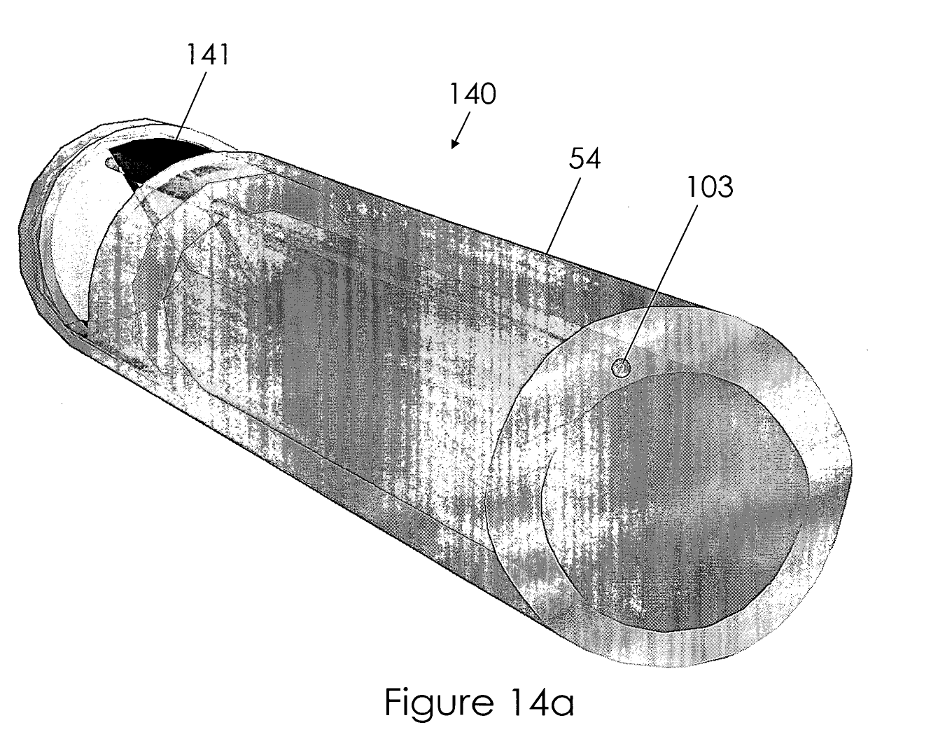

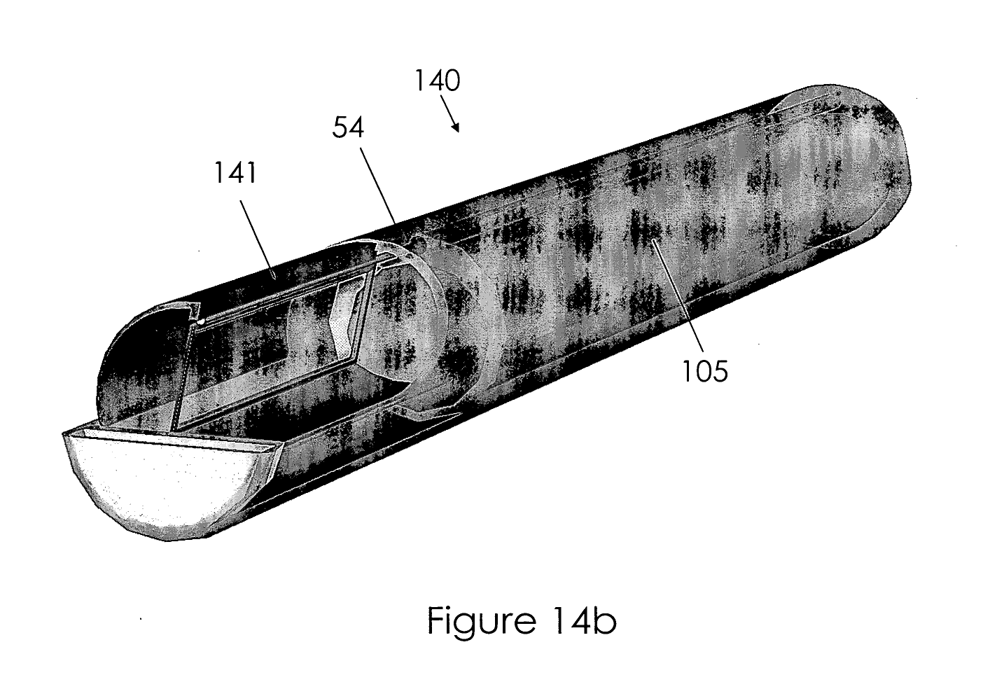

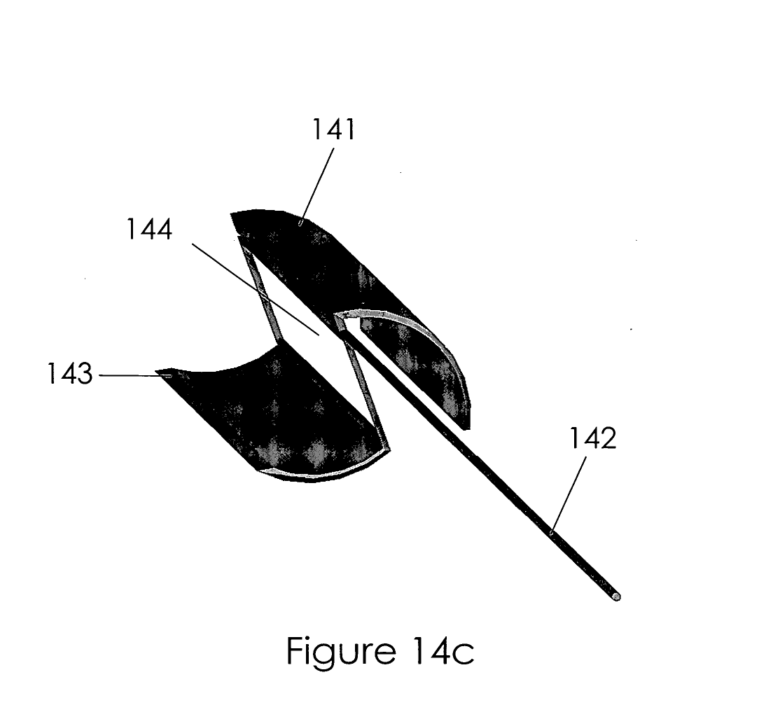

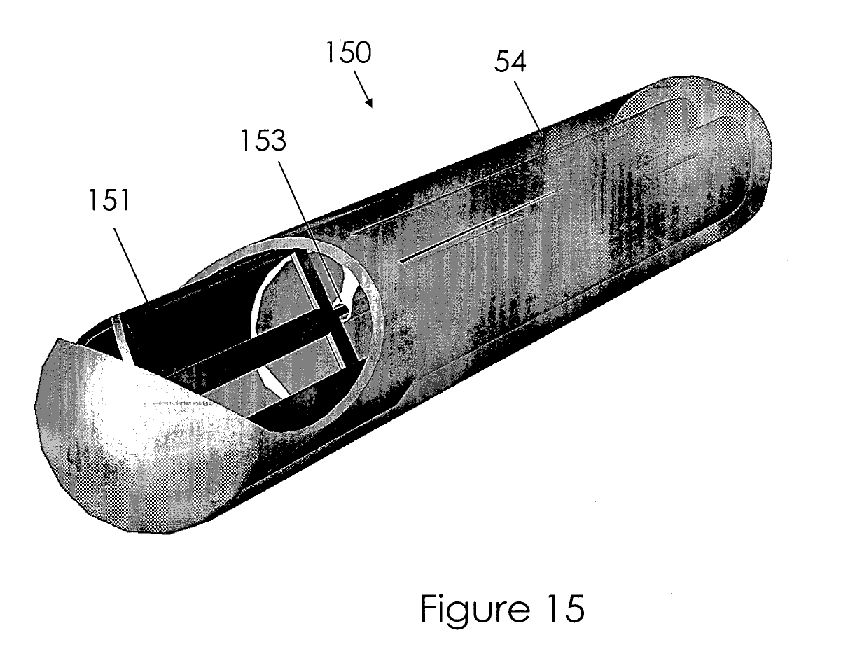

FIGS. 14a, 14b and 14c illustrate an embodiment of the present invention 140 with vanes 141 formed on the a rotatable control rod 142. The control rod passes through a lumen 103 formed in the wall of collection tube 54. When rotated, the vanes serve to disrupt or sever nucleus material 20 that enters an opening at the end of the collection tube 54. The length of the vanes is preferably selected to be able to substantially or completely occlude the opening in the collection tube through rotation and an opening 144 that approximates the cross section of the opening to permit the maximum amount of nucleus material 20 to enter the opening. As shown in FIG. 14c the vanes may comprise sharpened edge 143 to improve the ability to sever stiffened or granular nucleus material. The vanes may be rotated either in a single direction as circular motion or through 180 degrees and then returned to a starting position. Suction and/or an auger, as described above, serve to move entrained nucleus material proximally.

We claim:

1. A method for removing nucleus pulposus from a spine intervertebral disc comprising:
inserting a first hollow tube comprising at least one distal opening into the nucleus space of the intervertebral disc;
manipulating the first tube to engage nucleus material into the distal opening;
sliding a second hollow tube distally over the first tube to cover the distal opening and separate the nucleus material engaged within the distal opening from remaining nucleus material;
applying suction pressure to the proximal end of the first tube to move the engaged nucleus material proximally within the first tube;
retracting the second tube proximally to expose the distal opening.

2. The method of claim 1 wherein the steps are repeated until substantial all nucleus material is removed from the intervertebral disc.

3. A device for removing nucleus pulposus from an intervertebral disc comprising:
a first elongate member with a hollow lumen extending through the member;
a first opening formed in one side of the distal portion of said elongate member, the first opening in fluid communication with the lumen;
a second hollow elongate member slideably arranged around the first elongate member;
a cutting edge formed on the distal end of the second elongate member;
a controllable source of fluid pressure in fluid communication with the lumen of the first elongate member at the proximal end of the elongate member.

4. The device of claim 3 further comprising handles are attached near the proximal ends of the first and second elongate members.

5. The device of claim 3 further comprising a soft tip formed at the distal end of the first elongate member.

6. The device of claim 3 wherein the outside diameter of the second elongate tube is preferably between 4 and 7 mm.

7. The device of claim 3 wherein the diameter of the lumen of the first elongate member is preferably between 2.5 and 4 mm.
